(19) United States
(12) Patent Application Publication
(10) Pub. No.: US 2016/0022987 A1
(43) Pub. Date: Jan. 28, 2016

(54) DEVICE AND METHOD FOR THE APPLICATION OF A TRANSCUTANEOUS ELECTRICAL STIMULATION STIMULUS

(71) Applicant: cerbomed GmbH, Erlangen (DE)

(72) Inventors: Thomas ZSCHAECK, Nuernberg (DE); Wolf Gerhard FRENKEL, Inzigkofen-Engelswies (DE); Martin HYCA, Erlangen (DE); Andreas HARTLEP, Holzkirchen (DE)

(21) Appl. No.: 14/639,451

(22) Filed: Mar. 5, 2015

(30) Foreign Application Priority Data
Jul. 27, 2014 (DE) 10 2014 010 882.1

(51) Int. Cl. A61N 1/36; A61N 1/04

(52) U.S. Cl. CPC A61N 1/36021; A61N 1/0456

(57) ABSTRACT

A device for the application of a transcutaneous electric stimulation stimulus onto the surface of a section of the human ear, which comprises a number of electrodes, wherein the device comprises a controlling device which controlling device is designed for the application of a stimulation current which is applied via the electrodes according to a predetermined run of the stimulation current along the time. To allow an effective treatment of different diseases by means of a transcutaneous stimulation of the vagus nerve the invention proposes that the stimulation device creates a periodic recurrent stimulation current, wherein the frequency of the recurrent stimulation current is between 0.01 Hz and 10 Hz. Furthermore, the invention relates a method for the treatment of migraine.

### CROSS-REFERENCE TO RELATED APPLICATIONS

The present application claims priority of 10 2014 010 882.1, filed Jul. 27, 2014, the priority of this application is hereby claimed and this application is incorporated herein by reference.

### BACKGROUND OF THE INVENTION

The invention relates to a device for the application of a transcutaneous electric stimulation stimulus onto the surface of a section of the human ear, which comprises a number of electrodes, wherein the device comprises a controlling device which controlling device is designed for the application of a stimulation current which is applied via the electrodes according to a predetermined run of the stimulation current along the time. Furthermore, the invention relates a method for the treatment of migraine.

It is generally known to take influence on the neurophysiological and neuroelectrical quality through invasive and non-invasive stimulation of the nerves and thereby on the function of the simulated nerves. Hereby different conditions of sickness can be treated. Numerous devices exist both for the invasive and the non-invasive stimulation.

The present invention is basing upon the method of the transcutaneous electrical stimulation of the nerves. At this method pulse currents of different current forms, amplitudes, pulse durations and frequencies are administered through the skin on different nerves and change their status parameter in an advantageous way.

A device of the kind mentioned above is known from DE 10 2010 054 165 B3. Here, a device for the transcutaneous stimulation of the vagus nerve of the human body is described which electrode head with two electrodes is arranged in the region of the Cymba conchae; such a positioning of the electrodes has been proven as being beneficial. Thereby, the region of the Cymba conchae is that region of the concha of the ear which is located above the Crus helicis; it is also called

Hemiconcha superior. Then, below the Crus helicis downwards the region of the Cavum conchae extends.

Thereby, it is typical that periodic current sequences are applied onto the vagus nerve via the electrodes, wherein relatively high frequencies of at least 25 Hz are used, partially also significant higher frequencies.

Thereby, it was found out that for some diseases no satisfying result is obtained. Here, especially the treatment of migraine is concerned which is sometimes not efficiently treatable when using conventional stimulation devices.

### SUMMARY OF THE INVENTION

Thus, it is an object of the present invention, to supply a device of the above mentioned kind by which it is possible to allow an effective treatment of specific neurological diseases by means of the transcutaneous vagus nerve stimulation. Furthermore, a method for the effective treatment of migraine should be provided.

The solution of this object by the invention is characterized in that the stimulation device creates a periodic recurrent stimulation current, wherein the frequency of the recurrent stimulation current is between 0.01 Hz and 10 Hz.

The frequency of the recurrent stimulation current is thereby preferably between 0.1 Hz and 5 Hz, especially between 0.5 Hz and 2 Hz, and specifically preferred between 0.75 Hz and 1.25 Hz.

The periodically recurrent stimulation current is preferably biphasic, i.e. the current has positive and negative parts.

The stimulation device creates thereby preferably current pulses with a predetermined duration which are interrupted by current-free phases. The duration of the current pulses is thereby preferably between 100 μs and 1,000 μs, preferably between 200 μm and 300 μm.

The amplitude of the stimulation current is preferably between 0.02 mA and 5.0 mA.

The maximum values of the stimulation current of the current pulses can be increasing at least along a predetermined time. Consequently, the single current pulses increase according to a ramp function.

The course of the function of the current pulses is preferably rectangular or trapezoid.

A further embodiment proposes that the stimulation device does not apply a current onto the electrodes after the creation of a number of current pulses of a predetermined duration during a following predetermined pause time. Thereby, it is preferably provided that the time, in which the number of current pulses is created, and the time, in which no current is applied onto the electrodes, are in a relationship between 1:1 and 1:100. Thus, it can be provided that after a number of current pulses of the mentioned kind and with the mentioned frequency a stimulation-free pause time is kept. Thereby, a stimulation for example along 5 seconds can take place, whereupon a stimulation-free pause time of several minutes occurs (at a pause time of 8 minutes the mentioned relationship would be then for example about 1:100). The release of further current pulses can of course also take place for example directly by the patient who causes single bursts (see below) in due time.

The method according to the invention for the treatment of migraine at which a transcutaneous electric stimulation stimulus is applied by means of a number of electrodes onto the surface of a section of the ear of a patient, wherein a stimulation current is applied via the electrodes according to a predetermined run of the stimulation current along the time is characterized in that a periodic recurrent stimulation current is created, wherein the frequency of the recurrent stimulation current is between 0.01 Hz and 10 Hz.

The frequency of the recurrent stimulation current is thereby preferably between 0.1 Hz and 5 Hz, preferably between 0.5 Hz and 2 Hz, specifically preferred between 0.75 Hz and 1.25 Hz.

The recurrent stimulation current is preferably biphasic. Furthermore, an embodiment according to the invention provides that current pulses with a predetermined duration are created which are interrupted by current-free phases. The duration of the current pulses is thereby preferably between 100 μs and 1,000 μs, preferably between 200 μm and 300 μm. The amplitude of the stimulation current is preferably between 0.02 mA and 5.0 mA. The maximum values of the stimulation current of the current pulses are preferably increasing at least along a predetermined time. The course of the function of the current pulses is thereby preferably rectangular or trapezoid.

After the creation of a number of current pulses of a predetermined duration no current can be applied onto the electrodes along a following pause time. The time, in which the number of current pulses is created, and the time, in which no current is applied onto the electrodes, are preferably in a relationship between 1:1 and 1:100.

To sum up it can be said accordingly that the following transcutaneous stimulation current has been proven as being specifically beneficial to allow especially a therapy for migraine:

The pulse current (amplitude of stimulation current) is preferably between 0.02 mA and 5 mA, wherein a biphasic pulse is employed. The pulse width is preferably between 100 μs and 1,000 μs (preferably 250 μs), the frequency is between 0.01 Hz and 10 Hz (preferably 1 Hz).

Preferably, burst cycles (thus bundles of single current pulses) are also employed with an intermittent stimulation; the burst cycles can thereby occur intermittent with an increasing and decreasing stimulation.

It was found out that specific neurological diseases respond specifically good to a transcutaneous auricular applied stimulation current in a rather low frequency range. Here, primarily migraine should be mentioned as disease.

Insofar the invention relates also to a method for the treatment of migraine with the mentioned development of the transcutaneous stimulation current.

The various features of novelty which characterize the invention are pointed out with particularity in the claims annexed to and forming a part of the disclosure. For a better understanding of the invention, its operating advantages, specific objects attained by its use, reference should be had to the drawings and descriptive matter in which there are illustrated and described preferred embodiments of the invention.

### BRIEF DESCRIPTION OF THE DRAWING

In the drawing:

FIG. 1 shows schematically the course of the stimulation current over the time and

FIG. 2 shows an enlarged section of the course according to FIG. 1.

### DETAILED DESCRIPTION OF THE INVENTION

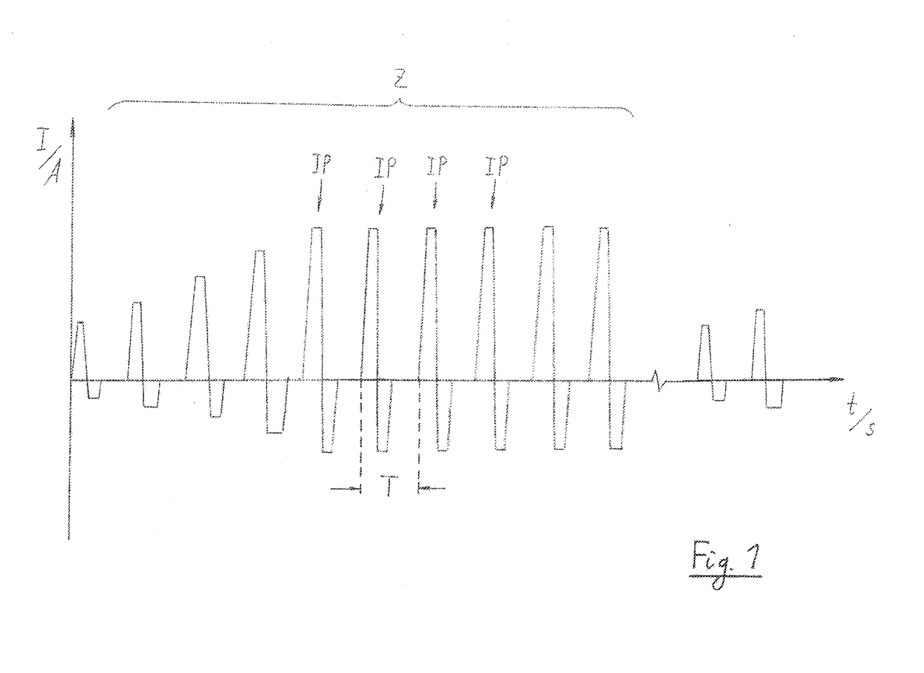

In FIG. 1 it is shown how the course of the stimulation current I over the time t can look like. At first, a cycle Z can be seen, wherein during the cycle Z a number of current pulses IP are released by control element of the stimulation device. The stimulation device needs not to be mentioned here any further as it is known as such sufficiently. Reference is made explicitly to DE 10 2010 054 165 B3 of the patent applicant with respect to pre-known devices which can be used with the present concept and where detailed information can be found. Accordingly, the device is designed to be arranged in the region of the vagus nerve at the ear of the person who uses the device. Thereby, a transcutaneous stimulation of the vagus nerve can be carried out.

In FIG. 1 it can be seen that after the cycle Z a pause time is kept after which a further cycle Z follows (respectively further cycles Z) from which however in FIG. 1 only the first two current pulses are depicted. The stimulation current I has a trapezoid course over the time t in the embodiment. Thereby, the periodically repeating single current pulses IP are distanced from another by a periodic time T so that a frequency f=1/T results for the stimulation which is according to the core of the proposed method preferably about 1 Hz (with the mentioned range).

It is not contradictory that the amplitudes of the stimulation current I along the single current pulses IP remain not necessarily equal. It can be—as depicted in FIG. 1—that a rising course, i. e. a “ramp-up”, takes place at the beginning of a cycle Z.

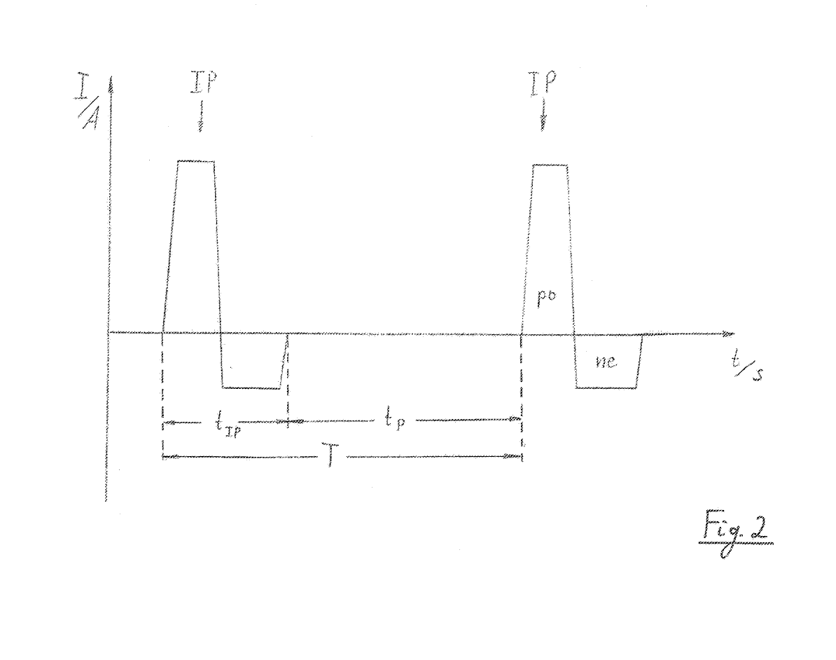

For two subsequent current pulses IP which have a stimulation current I of the same height an enlarged depiction is shown in FIG. 2. It can be seen that the biphasic designed current shape (i.e. beside a positive region po a negative region ne is provided) is trapezoid, wherein the stimulation current I flows along a duration t_{IP} of the current pulse. Then, a current-free phase follows which has a duration t_{P}. Thus, the whole pulse width of the current pulse IP is t_{IP}. From that, when regarding two subsequent current pulses IP, the periodic time T and from that in turn the frequency f=1/T can be determined which should be generally between 0.01 Hz and 10 Hz, however especially preferred at about 1 Hz.

As the preferred duration t_{IP} of a current pulse is about only 250 μs it can be seen that the depiction in the figures is not at scale at all.

While a trapezoid or rectangular course of the current is preferred, it can also be chosen in another manner. This can occur up to a sinusoidal course of the stimulation current which is then applied with the mentioned frequency via the electrodes.

Thus, while in the state of the art so far stimulation frequencies are used which are during the transcutaneous stimulation of the vagus nerve mostly in the range of milliseconds (for example with a pulse period of 40 ms, corresponding to a frequency of 25 Hz) the present idea proposes that other regions of the range are employed, namely pulse periods preferably of 1,000 ms, corresponding to a frequency of 1 Hz.

According to available studies, the mentioned range has shown a high efficiency with respect to the prophylactic treatment of chronic migraine.

While specific embodiments of the invention have been shown and described in detail to illustrate the inventive principles, it will be understood that the invention may be embodied otherwise without departing from such principles.

We claim:

1. A device for applying a transcutaneous electric stimulation stimulus onto a surface of a section of the human ear, the device comprising: a number of electrodes; a controlling device designed for applying a stimulation current which is applied via the electrodes according to a predetermined run of the stimulation current over time, wherein the device creates a periodic recurrent stimulation current, wherein the recurrent stimulation current has a frequency between 0.01 Hz and 10 Hz.

2. The device according to claim 1, wherein the frequency of the recurrent stimulation current is between 0.1 Hz and 5 Hz.

3. The device according to claim 2, wherein the frequency of the recurrent stimulation current is between 0.5 Hz and 2 Hz.

4. The device according to claim 3, wherein the frequency of the recurrent stimulation current is between 0.75 Hz and 1.25 Hz.

5. The device according to claim 1, wherein the recurrent stimulation current is biphasic.

6. The device according to claim 1, wherein the device creates current pulses with a predetermined duration which are interrupted by current-free phases.

7. The device according to claim 6, wherein the current pulses have a duration between 100 μs and 1,000 μs.

8. The device according to claim 7, wherein the current pulses have a duration between 200 μm and 300 μm.

9. The device according to claim 1, wherein the stimulation current has an amplitude between 0.02 mA and 5.0 mA.

10. The device according to claim 6, wherein maximum values of the stimulation current of the current pulses are increasing at least along a predetermined time.

11. The device according to claim 6, wherein the current pulses have a function with a course that is rectangular or trapezoidal.

12. The device according to claim 6, wherein the device does not apply a current onto the electrodes after creation of a number of current pulses of a predetermined duration during a following pause time.

13. The device according to claim 12, wherein a time, in which the number of current pulses is created, and the time, in which no current is applied onto the electrodes, are in a relationship between 1:1 and 1:100.

14. A method for treating migraine, comprising the steps of applying a transcutaneous electric stimulation stimulus by a number of electrodes onto a surface of a section of an ear of a patient; and applying a stimulation current via the electrodes according to a predetermined run of the stimulation current over time, wherein a periodic recurrent stimulation current is created, wherein the recurrent stimulation current has a frequency between 0.01 Hz and 10 Hz.

15. The method according to claim 14, wherein the frequency of the recurrent stimulation current is between 0.1 Hz and 5 Hz.

16. The method according to claim 15, wherein the frequency of the recurrent stimulation current is between 0.5 Hz and 2 Hz.

17. The method according to claim 16, wherein the frequency of the recurrent stimulation current is between 0.75 Hz and 1.25 Hz.

18. The method according to claim 14, wherein the recurrent stimulation current is biphasic.

19. The method according to claim 14, including creating current pulses with a predetermined duration, which are interrupted by current-free phases.

20. The method according to claim 19, wherein the current pulses have a duration between 100 μs and 1,000 μs.

21. The method according to claim 20, wherein the current pulses have a duration between 200 μm and 300 μm.

22. The method according to claim 14, wherein the stimulation current has an amplitude between 0.02 mA and 5.0 mA.

23. The method according to claim 19, wherein maximum values of the stimulation current of the current pulses are increasing at least along a predetermined time.

24. The method according to claim 19, wherein the current pulses have a function with a course that is rectangular or trapezoidal.

25. The method according to claim 19, wherein after creation of a number of current pulses of a predetermined duration no current is applied onto the electrodes along a following pause time.

26. The method according to claim 25, wherein a time, in which the number of current pulses is created, and the time, in which no current is applied onto the electrodes, are in a relationship between 1:1 and 1:100.
